(19) United States
(12) Patent Application Publication
(10) Pub. No.: US 2019/0274657 A1
(43) Pub. Date: Sep. 12, 2019

(54) COOPERATIVE GUIDE COMPONENTS FOR ELECTRICAL CABLE ATTACHMENT AND ASSOCIATED INTRALUMINAL DEVICES, SYSTEMS, AND METHODS

(71) Applicant: KONINKLIJKE PHILIPS N.V., EINDHOVEN (NL)

(72) Inventor: David Kenneth Wrolstad, Fallbrook, CA (US)

(21) Appl. No.: 16/335,337

(22) PCT Filed: Sep. 27, 2017
(86) PCT No.: PCT/EP2017/074559

§ 371 Date: Mar. 21, 2019

Related U.S. Application Data
(60) Provisional application No. 62/401,566, filed on Sep. 29, 2016.

(51) Int. Cl. A61B 8/12; A61B 8/00

(52) U.S. Cl. CPC A61B 8/12; A61B 8/42; A61B 2562/12; A61B 8/445; A61B 2562/028; A61B 8/4494

(57) ABSTRACT

An intraluminal imaging device (102) includes a flexible elongate member (15) sized and shaped for insertion into a vessel of a patient, the flexible elongate member including a proximal portion and a distal portion; an imaging assembly (110) disposed at the distal portion of the flexible elongate member; a plurality of wires (330) extending along a length of the flexible elongate member and in communication with the imaging assembly; and a guide member (350) positioned at the proximal portion of the imaging assembly and comprising a plurality of conductive members, wherein the plurality of wires are in communication with the imaging assembly via the plurality of conductive members. Associated devices, systems, and methods are also provided.

### TECHNICAL FIELD

The present disclosure relates generally to intraluminal imaging and, in particular, to an imaging assembly of an intraluminal imaging device. For example, wires of an electrical cable can extend through conductive members that are received within a guide member positioned at a proximal portion of an imaging assembly. The cooperating conductive members and guide member can improve manufacturing efficiency by removing a soldering/welding step in some instances.

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. An IVUS device including one or more ultrasound transducers is passed into the vessel and guided to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed.

Solid-state (also known as synthetic-aperture) IVUS catheters are one of the two types of IVUS devices commonly used today, the other type being the rotational IVUS catheter. Solid-state IVUS catheters carry a scanner assembly that includes an array of ultrasound transducers distributed around its circumference along with one or more integrated circuit controller chips mounted adjacent to the transducer array. The controllers select individual transducer elements (or groups of elements) for transmitting an ultrasound pulse and for receiving the ultrasound echo signal. By stepping through a sequence of transmit-receive pairs, the solid-state IVUS system can synthesize the effect of a mechanically scanned ultrasound transducer but without moving parts (hence the solid-state designation). Since there is no rotating mechanical element, the transducer array can be placed in direct contact with the blood and vessel tissue with minimal risk of vessel trauma. Furthermore, because there is no rotating element, the electrical interface is simplified. The solid-state scanner can be wired directly to the imaging system with a simple electrical cable and a standard detachable electrical connector, rather than the complex rotating electrical interface required for a rotational IVUS device.

The electrical cable and the solid-state scanner are connected during assembly of the IVUS device. Generally, this requires that conductors in the electrical cable be individually aligned with a respective conductive pad on the solid-state scanner. The electrical cable includes many different conductors. The conductors can be individually soldered/welded to the respective conductive pads, which electrically couples the electrical couples the electrical cable and the solid-state scanner. Accordingly, individually aligning and connecting the conductors and the conductive pads can be a time-consuming step during manufacturing.

### SUMMARY

The invention provides imaging devices, systems, and related methods that overcome the limitations associated with individually aligning and soldering conductors of imaging assemblies.

Embodiments of the present disclosure provide an improved intraluminal imaging system for generating images of a blood vessel. Wires of an electrical cable are attached to respective conductive members. The conductive members positioned around a guide member with recesses that are sized and shaped to accommodate the conductive members. The guide member, along with the wires, can be attached to the imaging assembly. A conductor interface of the imaging assembly can be wrapped around the guide member so that conductive pads of the conductor interface contact respective conductive members. The wires, conductive members, the guide member, and/or the conductor interface can be attached without soldering/welding. Manufacturing efficiency is improved by avoiding the need to individually align and solder/weld the wires to the conductive pads of the imaging assembly.

In one embodiment, an intraluminal imaging device is provided. The intraluminal imaging devices includes a flexible elongate member configured for insertion into a vessel of a patient, the flexible elongate member including a proximal portion and a distal portion; an imaging assembly disposed at the distal portion of the flexible elongate member; a plurality of wires extending along a length of the flexible elongate member and in communication with the imaging assembly; and a guide member being at a proximal portion of the imaging assembly and comprising a plurality of conductive members, wherein the plurality of wires are in communication with the imaging assembly via the plurality of conductive members.

In some embodiments, the imaging assembly comprises a conductor interface; and the conductor interface is positioned around at least a portion of the guide member. In some embodiments, the conductor interface comprises a plurality of conductive pads, and each of the plurality of conductive pads is in contact with a respective one of the plurality of conductive members of the guide member when the conductor interface is positioned around the guide member. In some embodiments, the imaging assembly comprises a support member and a flex circuit positioned at least partially around the support member, and the conductor interface extends from a proximal portion of the flex circuit. In some embodiments, the guide member is annular. In some embodiments, the guide member is positioned around a proximal portion of the support member. In some embodiments, each of the plurality of conductive members comprises a bore configured to receive at least one of the plurality of wires. In some embodiments, each of the plurality of wires extends through the bore of a respective one of the plurality of conductive members. In some embodiments, the plurality of conductive members and the guide member are distinct components that are coupled. In some embodiments, the guide member comprises a plurality of recesses, each of the plurality of recesses configured to receive a respective one of the plurality of conductive members. In some embodiments, the intraluminal imaging device includes an engagement mechanism positioned adjacent to the guide member and the plurality of conductive members, and arranged to maintain to plurality of conductive members within the plurality of recesses of the guide member. In some embodiments, the plurality of conductive members is positioned around a circumference of the guide member. In some embodiments, the guide member comprises non-conductive material.

In one embodiment, a method of assembling an intraluminal imaging device is provided. The method includes obtaining an imaging assembly comprising a guide member; coupling each of a plurality of wires to a respective one of a plurality of conductive members; and coupling the plurality of wires to the imaging assembly by positioning the plurality of conductive members around the guide member.

In some embodiments, the imaging assembly comprises a conductor interface, the method further comprising: positioning the conductor interface around the guide member. In some embodiments, the conductor interface comprises a plurality of conductive pads, and wherein the positioning the conductor interface around the guide member comprises contacting each of the plurality of conductive pads of the conductor interface with a respective one of the plurality of conductive members. In some embodiments, the imaging assembly comprises a support member and a flex circuit positioned around the support member, wherein the guide member is annular, and wherein the method further comprising: positioning the guide member around a proximal portion of the support member. In some embodiments, the guide member comprises a plurality of recesses, wherein the positioning the plurality of conductive members around the guide member comprises positioning each of the plurality of conductive members within a respective one of the plurality of recesses. In some embodiments, the method includes positioning an engagement mechanism adjacent to the guide member and the plurality of conductive members to maintain to plurality of conductive members within the plurality of recesses of the guide member. In some embodiments, each of the plurality of conductive members comprises a bore, and wherein the coupling each of a plurality of wires to a respective one of a plurality of conductive members comprises inserting each of the plurality of wires into the bore of the respective one of the plurality of conductive members.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an imaging system, according to aspects of the present disclosure.

FIG. 2 is a diagrammatic top view of a scanner assembly in a flat configuration, according to aspects of the present disclosure.

FIG. 3 is a diagrammatic side view of a scanner assembly in a rolled configuration around a support member, according to aspects of the present disclosure.

FIG. 4 is a diagrammatic cross-sectional side view of a distal portion of an intraluminal device, according to aspects of the present disclosure.

FIG. 5 is a diagrammatic perspective view of an imaging assembly coupled to an electrical cable, according to aspects of the present disclosure.

FIG. 6 is a cross-sectional back view of an imaging assembly coupled to an electrical cable, according to aspects of the present disclosure.

FIG. 7 is a cross-sectional side view an imaging assembly coupled to an electrical cable, according to aspects of the present disclosure.

FIG. 8 is a diagrammatic perspective view of a guide member, according to aspects of the present disclosure.

FIG. 9 is a diagrammatic perspective view of a conductive member, according to aspects of the present disclosure.

FIG. 10 is a cross-sectional side view an imaging assembly coupled to an electrical cable, according to aspects of the present disclosure.

FIG. 11 is a cross-sectional side view an imaging assembly coupled to an electrical cable, according to aspects of the present disclosure.

FIG. 12 is a cross-sectional side view an imaging assembly coupled to an electrical cable, according to aspects of the present disclosure.

FIG. 13 is a cross-sectional side view an imaging assembly coupled to an electrical cable, according to aspects of the present disclosure.

FIG. 14 is a flow diagram of a method of assembly an intraluminal imaging device, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

The present disclosure describes an imaging assembly for an intraluminal imaging device. The imaging assembly includes a flex circuit positioned at a distal portion of a flexible elongate member. A conductor interface having conductive pads extends from a proximal portion of the flex circuit. Wires of an electrical cable are coupled to respective conductive members. For example, the wires extend through bores of the conductive members. The conductive members can be positioned within recesses of a guide member. The guide member, along with the wires, can be coupled to the imaging assembly to facilitate electrical communication between the imaging assembly and the wires. For example, the conductor interface of the flex circuit can be wrapped around the guide member such that the conductive pads contact respective conductive members.

The intraluminal imaging device described herein achieves numerous advantages. For example, the guide member and the conductive members facilitate a faster, less labor-intensive, and more efficient manufacturing process for the intraluminal device. In that regard, rather than individually soldering/welding the wires to the respective conductive pads, the wires, conductive members, the guide member, and/or the conductor interface can be attached via any suitable engaging fit and/or adhesive(s). The wires and the conductive members can be formed of conductive materials to facilitate electrical signal transfer.

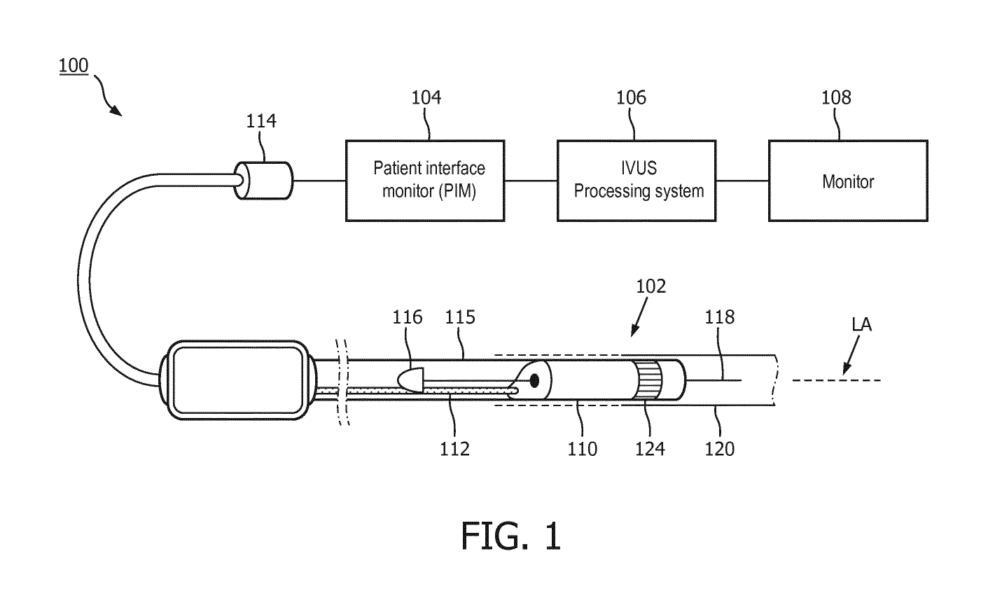

FIG. 1 is a diagrammatic schematic view of an intravascular ultrasound (IVUS) imaging system 100, according to aspects of the present disclosure. The IVUS imaging system 100 may include a solid-state IVUS device 102 such as a catheter, guide wire, or guide catheter, a patient interface module (PIM) 104, an IVUS processing system or console 106, and a monitor 108.

At a high level, the IVUS device 102 emits ultrasonic energy from a transducer array 124 included in scanner assembly 110 mounted near a distal end of the catheter device. The ultrasonic energy is reflected by tissue structures in the medium, such as a vessel 120, surrounding the scanner assembly 110, and the ultrasound echo signals are received by the transducer array 124. The PIM 104 transfers the received echo signals to the console or computer 106 where the ultrasound image (including the flow information) is reconstructed and displayed on the monitor 108. The console or computer 106 can include a processor and a memory. The computer or computing device 106 can be operable to facilitate the features of the IVUS imaging system 100 described herein. For example, the processor can execute computer readable instructions stored on the non-transitory tangible computer readable medium.

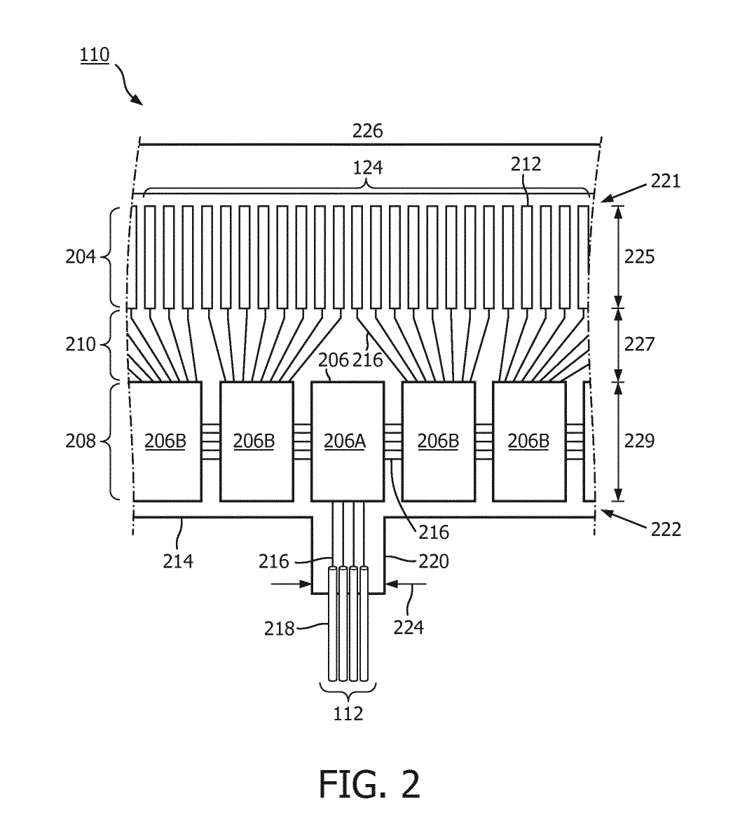

The PIM 104 facilitates communication of signals between the IVUS console 106 and the scanner assembly 110 included in the IVUS device 102. This communication includes the steps of: (1) providing commands to integrated circuit controller chip(s) 206A, 206B, illustrated in FIG. 2, included in the scanner assembly 110 to select the particular transducer array element(s) to be used for transmit and receive, (2) providing the transmit trigger signals to the integrated circuit controller chip(s) 206A, 206B included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) 126 of the scanner assembly 110. In some embodiments, the PIM 104 performs preliminary processing of the echo data prior to relaying the data to the console 106. In examples of such embodiments, the PIM 104 performs amplification, filtering, and/or aggregating of the data. In an embodiment, the PIM 104 also supplies high- and low-voltage DC power to support operation of the device 102 including circuitry within the scanner assembly 110.

The IVUS console 106 receives the echo data from the scanner assembly 110 by way of the PIM 104 and processes the data to reconstruct an image of the tissue structures in the medium surrounding the scanner assembly 110. The console 106 outputs image data such that an image of the vessel 120, such as a cross-sectional image of the vessel 120, is displayed on the monitor 108. Vessel 120 may represent fluid filled or surrounded structures, both natural and man-made. The vessel 120 may be within a body of a patient. The vessel 120 may be a blood vessel, as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be may be used to examine man-made structures such as, but without limitation, heart valves, stents, shunts, filters and other devices.

In some embodiments, the IVUS device includes some features similar to traditional solid-state IVUS catheters, such as the EagleEye® catheter available from Volcano Corporation and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors 218 (FIG. 2). It is understood that any suitable gauge wire can be used for the conductors 218. In an embodiment, the cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a PIM connector 114 at a proximal end of the device 102. The PIM connector 114 electrically couples the transmission line bundle 112 to the PIM 104 and physically couples the IVUS device 102 to the PIM 104. In an embodiment, the IVUS device 102 further includes a guide wire exit port 116. Accordingly, in some instances the IVUS device is a rapid-exchange catheter. The guide wire exit port 116 allows a guide wire 118 to be inserted towards the distal end in order to direct the device 102 through the vessel 120.

The IVUS device 102 includes a flexible elongate member 115 having a proximal portion and a distal portion. The scanner assembly 110 is positioned at a distal portion of the flexible elongate member 115. The flexible elongate member 115 includes a longitudinal axis LA. The longitudinal axis LA may be associated with the IVUS device 102 and/or the imaging assembly 110.

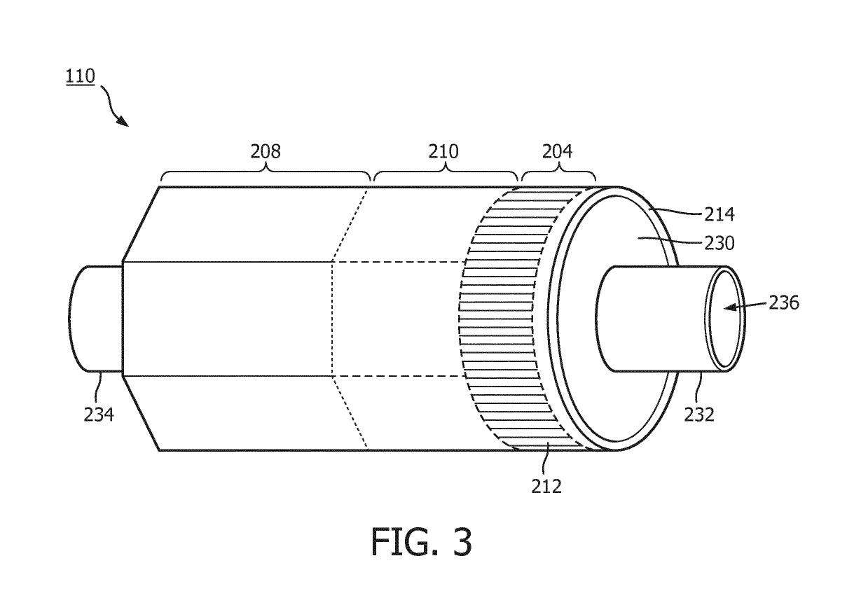

FIG. 2 is a top view of a portion of an ultrasound scanner assembly 110 according to an embodiment of the present disclosure. The assembly 110 includes a transducer array 124 formed in a transducer region 204 and transducer control logic dies 206 (including dies 206A and 206B) formed in a control region 208, with a transition region 210 disposed therebetween. The transducer control logic dies 206 and the transducers 212 are mounted on a flex circuit 214 that is shown in a flat configuration in FIG. 2. FIG. 3 illustrates a rolled configuration of the flex circuit 214. The transducer array 202 is a non-limiting example of a medical sensor element and/or a medical sensor element array. The transducer control logic dies 206 is a non-limiting example of a control circuit. The transducer region 204 is disposed adjacent a distal portion 221 of the flex circuit 214. The control region 208 is disposed adjacent the proximal portion 222 of the flex circuit 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or a length 227 of the transition region 210 can be greater than lengths 225, 229 of the transducer region and controller region, respectively. While the imaging assembly 110 is described as including a flex circuit, it is understood that the transducers and/or controllers may be arranged to form the imaging assembly 110 in other configurations, including those omitting a flex circuit.

The transducer array 124 may include any number and type of ultrasound transducers 212, although for clarity only a limited number of ultrasound transducers are illustrated in FIG. 2. In an embodiment, the transducer array 124 includes 64 individual ultrasound transducers 212. In a further embodiment, the transducer array 124 includes 32 ultrasound transducers 212. Other numbers are both contemplated and provided for. With respect to the types of transducers, in an embodiment, the ultrasound transducers 212 are piezoelectric micromachined ultrasound transducers (PMUTs) fabricated on a microelectromechanical system (MEMS) substrate using a polymer piezoelectric material, for example as disclosed in U.S. Pat. No. 6,641,540, which is hereby incorporated by reference in its entirety. In alternate embodiments, the transducer array includes piezoelectric zirconate transducers (PZT) transducers such as bulk PZT transducers, capacitive micromachined ultrasound transducers (cMUTs), single crystal piezoelectric materials, other suitable ultrasound transmitters and receivers, and/or combinations thereof.

The scanner assembly 110 may include various transducer control logic, which in the illustrated embodiment is divided into discrete control logic dies 206. In various examples, the control logic of the scanner assembly 110 performs: decoding control signals sent by the PIM 104 across the cable 112, driving one or more transducers 212 to emit an ultrasonic signal, selecting one or more transducers 212 to receive a reflected echo of the ultrasonic signal, amplifying a signal representing the received echo, and/or transmitting the signal to the PIM across the cable 112. In the illustrated embodiment, a scanner assembly 110 having 64 ultrasound transducers 212 divides the control logic across nine control logic dies 206, of which five are shown in FIG. 2. Designs incorporating other numbers of control logic dies 206 including 8, 9, 16, 17 and more are utilized in other embodiments. In general, the control logic dies 206 are characterized by the number of transducers they are capable of driving, and exemplary control logic dies 206 drive 4, 8, and/or 16 transducers.

The control logic dies are not necessarily homogenous. In some embodiments, a single controller is designated a master control logic die 206A and contains the communication interface for the cable 112. Accordingly, the master control circuit may include control logic that decodes control signals received over the cable 112, transmits control responses over the cable 112, amplifies echo signals, and/or transmits the echo signals over the cable 112. The remaining controllers are slave controllers 206B. The slave controllers 206B may include control logic that drives a transducer 212 to emit an ultrasonic signal and selects a transducer 212 to receive an echo. In the depicted embodiment, the master controller 206A does not directly control any transducers 212. In other embodiments, the master controller 206A drives the same number of transducers 212 as the slave controllers 206B or drives a reduced set of transducers 212 as compared to the slave controllers 206B. In an exemplary embodiment, a single master controller 206A and eight slave controllers 206B are provided with eight transducers assigned to each slave controller 206B.

The flex circuit 214, on which the transducer control logic dies 206 and the transducers 212 are mounted, provides structural support and interconnects for electrical coupling. The flex circuit 214 may be constructed to include a film layer of a flexible polyimide material such as KAPTON™ (trademark of DuPont). Other suitable materials include polyester films, polyimide films, polyethylene napthalate films, or polyetherimide films, other flexible printed semiconductor substrates as well as products such as Upilex® (registered trademark of Ube Industries) and TEFLON® (registered trademark of E.I. du Pont). In the flat configuration illustrated in FIG. 2, the flex circuit 214 has a generally rectangular shape. As shown and described herein, the flex circuit 214 is configured to be wrapped around a support member 230 (FIG. 3) to form a cylindrical toroid in some instances. Therefore, the thickness of the film layer of the flex circuit 214 is generally related to the degree of curvature in the final assembled scanner assembly 110. In some embodiments, the film layer is between 5 μm and 100 μm, with some particular embodiments being between 12.7 μm and 25.1 μm.

To electrically interconnect the control logic dies 206 and the transducers 212, in an embodiment, the flex circuit 214 further includes conductive traces 216 formed on the film layer that carry signals between the control logic dies 206 and the transducers 212. In particular, the conductive traces 216 providing communication between the control logic dies 206 and the transducers 212 extend along the flex circuit 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 112 when the conductors 218 of the cable 112 are mechanically and electrically coupled to the flex circuit 214. Suitable materials for the conductive traces 216 include copper, gold, aluminum, silver, tantalum, nickel, and tin, and may be deposited on the flex circuit 214 by processes such as sputtering, plating, and etching. In an embodiment, the flex circuit 214 includes a chromium adhesion layer. The width and thickness of the conductive traces 216 are selected to provide proper conductivity and resilience when the flex circuit 214 is rolled. In that regard, an exemplary range for the thickness of a conductive trace 216 and/or conductive pad is between 10-50 μm. For example, in an embodiment, 20 μm conductive traces 216 are separated by 20 μm of space. The width of a conductive trace 216 on the flex circuit 214 may be further determined by the width of the conductor 218 to be coupled to the trace/pad.

The flex circuit 214 can include a conductor interface 220 in some embodiments. The conductor interface 220 can be a location of the flex circuit 214 where the conductors 218 of the cable 112 are coupled to the flex circuit 214. For example, the bare conductors of the cable 112 are electrically coupled to the flex circuit 214 at the conductor interface 220. The conductor interface 220 can be tab extending from the main body of flex circuit 214. In that regard, the main body of the flex circuit 214 can refer collectively to the transducer region 204, controller region 208, and the transition region 210. In the illustrated embodiment, the conductor interface 220 extends from the proximal portion 222 of the flex circuit 214. In other embodiments, the conductor interface 220 is positioned at other parts of the flex circuit 214, such as the distal portion 221, or the flex circuit 214 omits the conductor interface 220. A value of a dimension of the tab or conductor interface 220, such as a width 224, can be less than the value of a dimension of the main body of the flex circuit 214, such as a width 226. In some embodiments, the substrate forming the conductor interface 220 is made of the same material(s) and/or is similarly flexible as the flex circuit 214. In other embodiments, the conductor interface 220 is made of different materials and/or is comparatively more rigid than the flex circuit 214. For example, the conductor interface 220 can be made of a plastic, thermoplastic, polymer, hard polymer, etc., including polyoxymethylene (e.g., DELRIN®), polyether ether ketone (PEEK), nylon, and/or other suitable materials. As described in greater detail herein, the support member 230, the flex circuit 214, the conductor interface 220 and/or the conductor(s) 218 can be variously configured to facilitate efficient manufacturing and operation of the scanner assembly 110.

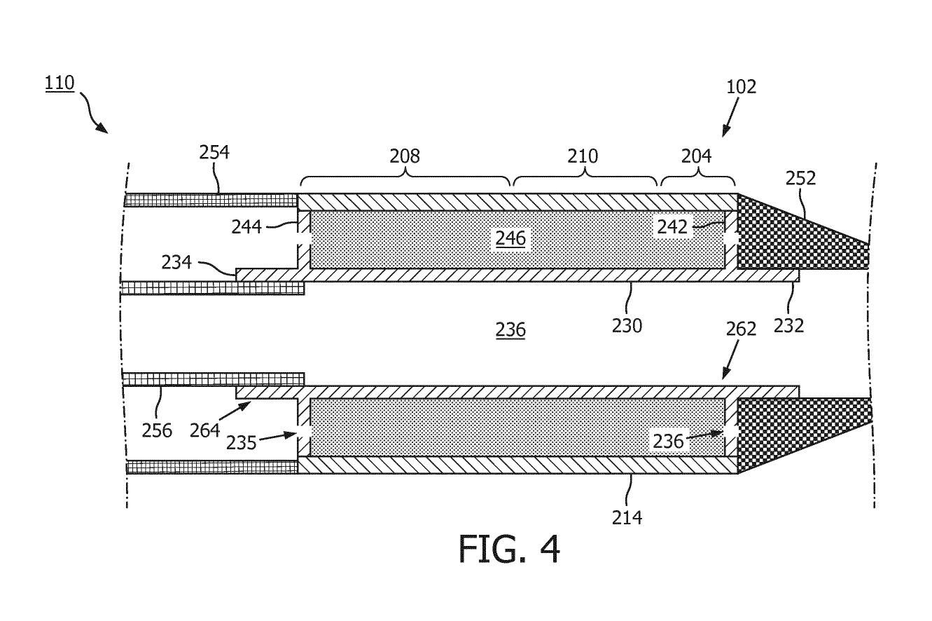

In some instances, the scanner assembly 110 is transitioned from a flat configuration (FIG. 2) to a rolled or more cylindrical configuration (FIGS. 3 and 4). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND TRANSDUCER ASSEMBLY HAVING A FLEXIBLE SUBSTRATE,” each of which is hereby incorporated by reference in its entirety.

As shown in FIGS. 3 and 4, the flex circuit 214 is positioned around the support member 230 in the rolled configuration. FIG. 3 is a diagrammatic side view with the flex circuit 214 in the rolled configuration around the support member 230, according to aspects of the present disclosure. FIG. 4 is a diagrammatic cross-sectional side view of a distal portion of the intraluminal device 102, including the flex circuit 214 and the support member 230, according to aspects of the present disclosure.

The support member 230 can be referenced as a unibody in some instances. The support member 230 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, the entirety of which is hereby incorporated by reference herein. The support member 230 can be ferrule having a distal portion 262 and a proximal portion 264. The support member 230 can define a lumen 236 extending longitudinally therethrough. The lumen 236 is in communication with the exit port 116 and is sized and shaped to receive the guide wire 118 (FIG. 1). The support member 230 can be manufactured accordingly to any suitable process. For example, the support member 230 can be machined, such as by removing material from a blank to shape the support member 230, or molded, such as by an injection molding process. In some embodiments, the support member 230 may be integrally formed as a unitary structure, while in other embodiments the support member 230 may be formed of different components, such as a ferrule and stands 242, 244, that are fixedly coupled to one another.

Stands 242, 244 that extend vertically are provided at the distal and proximal portions 262, 264, respectively, of the support member 230. The stands 242, 244 elevate and support the distal and proximal portions of the flex circuit 214. In that regard, portions of the flex circuit 214, such as the transducer portion 204, can be spaced from a central body portion of the support member 230 extending between the stands 242, 244. The stands 242, 244 can have the same outer diameter or different outer diameters. For example, the distal stand 242 can have a larger or smaller outer diameter than the proximal stand 244. To improve acoustic performance, any cavities between the flex circuit 214 and the surface of the support member 230 are filled with a backing material 246. The liquid backing material 246 can be introduced between the flex circuit 214 and the support member 230 via passageways 235 in the stands 242, 244. In some embodiments, suction can be applied via the passageways 235 of one of the stands 242, 244, while the liquid backing material 246 is fed between the flex circuit 214 and the support member 230 via the passageways 235 of the other of the stands 242, 244. The backing material can be cured to allow it to solidify and set. In various embodiments, the support member 230 includes more than two stands 242, 244, only one of the stands 242, 244, or neither of the stands. In that regard the support member 230 can have an increased diameter distal portion 262 and/or increased diameter proximal portion 264 that is sized and shaped to elevate and support the distal and/or proximal portions of the flex circuit 214.

The support member 230 can be substantially cylindrical in some embodiments. Other shapes of the support member 230 are also contemplated including geometrical, non-geometrical, symmetrical, non-symmetrical, cross-sectional profiles. Different portions the support member 230 can be variously shaped in other embodiments. For example, the proximal portion 264 can have a larger outer diameter than the outer diameters of the distal portion 262 or a central portion extending between the distal and proximal portions 262, 264. In some embodiments, an inner diameter of the support member 230 (e.g., the diameter of the lumen 236) can correspondingly increase or decrease as the outer diameter changes. In other embodiments, the inner diameter of the support member 230 remains the same despite variations in the outer diameter.

A proximal inner member 256 and a proximal outer member 254 are coupled to the proximal portion 264 of the support member 230. The proximal inner member 256 and/or the proximal outer member 254 can be flexible elongate member that extend from proximal portion of the intraluminal device 102, such as the proximal connector 114, to the imaging assembly 110. For example, the proximal inner member 256 can be received within a proximal flange 234. The proximal outer member 254 abuts and is in contact with the flex circuit 214. A distal member 252 is coupled to the distal portion 262 of the support member 230. The distal member 252 can be a flexible component that defines a distal most portion of the intraluminal device 102. For example, the distal member 252 is positioned around the distal flange 232. The distal member 252 can abut and be in contact with the flex circuit 214 and the stand 242. The distal member 252 can be the distal-most component of the intraluminal device 102.

One or more adhesives can be disposed between various components at the distal portion of the intraluminal device 102. For example, one or more of the flex circuit 214, the support member 230, the distal member 252, the proximal inner member 256, and/or the proximal outer member 254 can be mechanically coupled to one another via an adhesive.

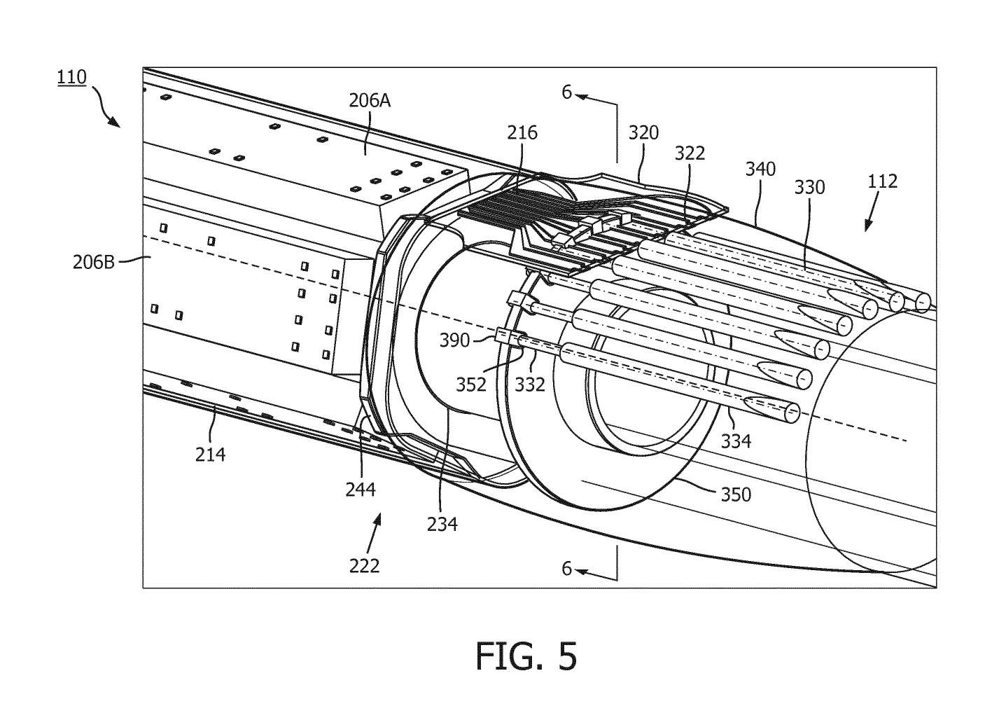

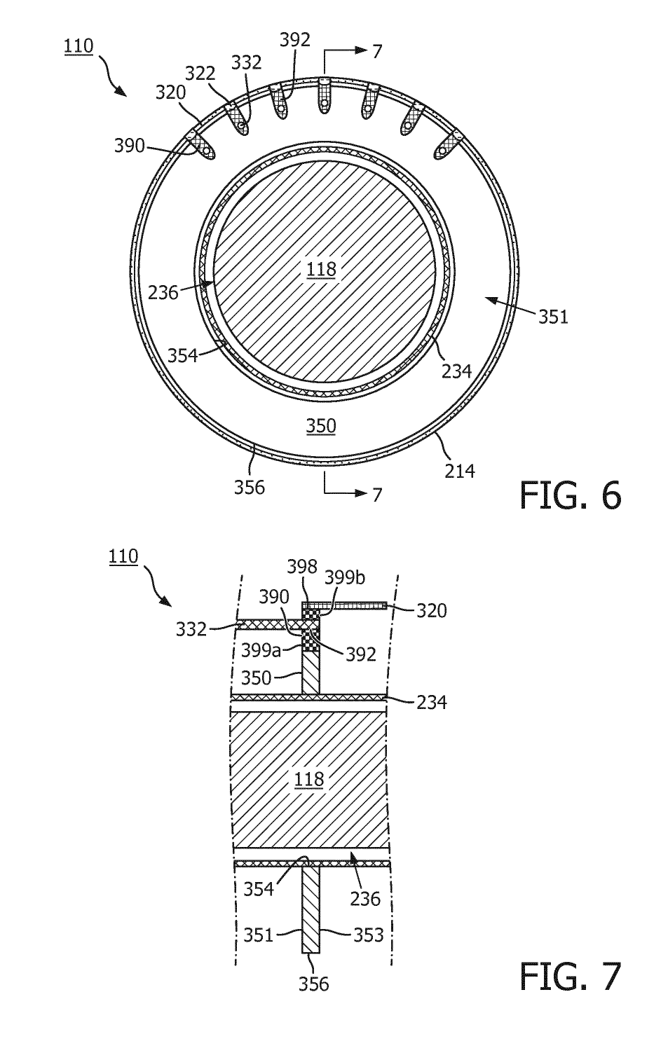

FIG. 5 is a diagrammatic perspective view of the distal portion of the intraluminal device 102, and more specifically, the proximal portion of the imaging assembly 110. FIG. 6 is a cross-sectional view back view of the proximal portion of the imaging assembly 110 along section line 6-6 of FIG. 5. FIG. 7 illustrates a cross-sectional side view of the proximal portion of the imaging assembly 110 along section line 7-7 of FIG. 6. FIGS. 6 and 7 generally show similar features of the imaging assembly 110 as FIG. 5. FIGS. 6 and 7 additionally illustrate the guide wire 118 extending through the lumen 236.

Referring generally to FIGS. 5, 6, and 7, shown is an exemplary embodiment of the imaging assembly 110 in which the flex circuit 214 is in electrical communication with the wires 330 of the cable 112 via a guide member 350 and conductive members 390. Generally, the guide member 350 and the conductive members 390 can be cooperative components that facilitate alignment and attachment of the cable 112 and the imaging assembly 110. Each of the wires 330 is electrically coupled to a respective conductive member 390. The conductive members 390 are positioned within recesses 352 of the guide member 350. In the illustrated embodiment, the guide member 350 is an annularly-shaped or ring-shaped component that is positioned around the proximal flange 234 of the support member 230. The conductor interface 320 of the flex circuit 214 can be positioned around the guide member 350 to establish electrical communication between the wires 330 and the flex circuit 214, the controllers 206A, 206B, and the transducer elements 212. For example, when the conductor interface 320 is positioned around the guide member 350, electrical communication is established by contact between respective conductive pads 322 and conductive members 390.

In the illustrated embodiment, the electrical cable 112 includes seven wires 330. It is understood that in other embodiments, the electrical cable 112 may have any suitable number of wires 330, including two, three, four, five, six, seven, eight, or more. Each wire 330 can have any suitable structure. For example, the wires 330 can include a bare conductor 332 surrounded by an insulation layer 334, as shown. In some embodiments, individual wires 330 and/or groupings of wires 330 can be further surrounded by additional insulation layer(s) or jacket(s). A distal portion of the electrical cable 112 is shown in FIGS. 5, 6, and 7. The wires 330 of the electrical cable 112 extend along a length of the flexible elongate member 115 of the intraluminal device 102 (FIG. 1), such as from the connector 114 at the proximal portion to the imaging assembly 110 at the distal portion.

As shown in FIGS. 5, 6, and 7, the conductors 332 are mechanically and electrically coupled to the conductive members 390. Aspects of exemplary conductive member 390 are shown and described with respect to FIG. 9. For example, the conductors 332 can extend through a bore 392 of the conductive members 390. The distal tip of the conductors 332 can be longitudinally aligned with or extend beyond the distal portion of the conductive members 390. The conductors 332 and the conductive members 390 can be made of conductive materials. Direct and/or indirect contact between the conductors 332 and the conductive members 390 establishes electrical communication. The conductors 332 can be press fit, interference fit, and/or otherwise engagingly fit within the bores 392. The conductors 332 may be soldered and/or welded to the conductive members 390 in some instances. In some instances, the conductors 332 may be crimped such that they are retained within the bores 392 of the conductive members 390. In some embodiments, adhesive(s) can be used to use to affix the conductors 332 within the conductive members 390. The adhesive(s) may be electrically conductive to facilitate electrical communication between the conductors 332 within the conductive members 390.

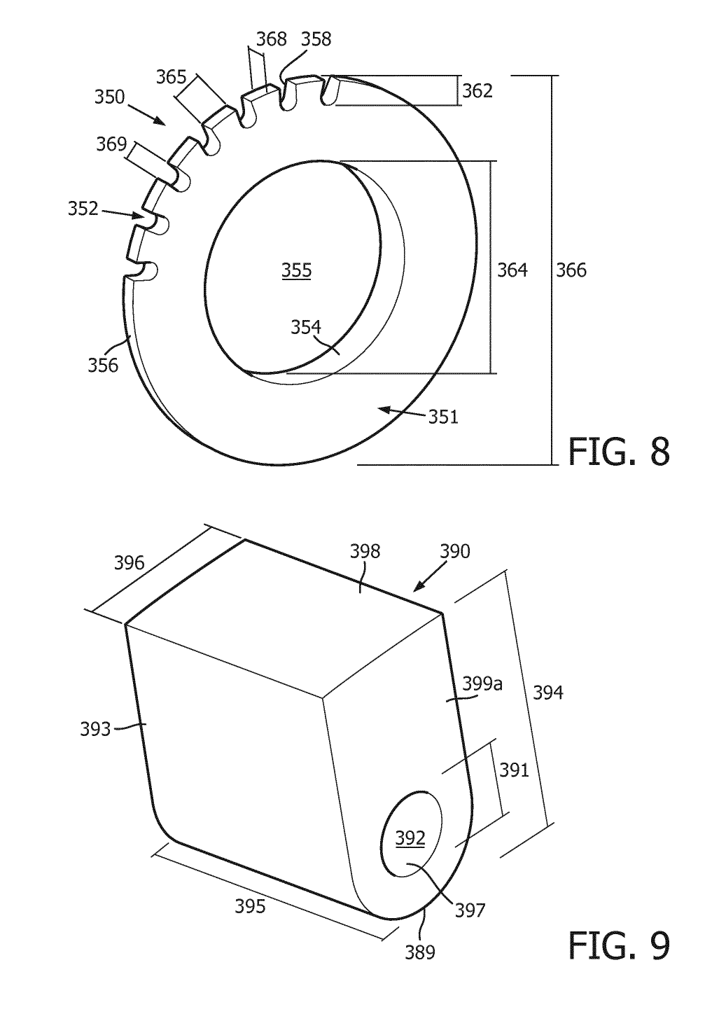

Referring to FIGS. 5, 6, and 7, the conductive members 390 are mechanically coupled to the guide member 350. Aspects of exemplary guide member 350 are shown and described with respect to FIG. 8. For example, each of the conductive members 390 can be positioned within a respective recess 352 of the guide member 350. The guide member 350 and the conductive members 390 can be distinct components such that the conductive members 390 are selectively positioned within the recesses 352 of the guide member 350. In some instances, the conductive members 390 are removably positioned within the guide member 350. In some instances, the conductive members 390 can be press fit, interference fit, and/or otherwise engagingly fit within the recesses 352. In some embodiments, adhesive(s) can be used to use to affix the conductive members 390 within the recesses 352. The guide member 350 can be formed of a non-conductive material such that electrical signals pass back and forth between the transducer elements 212, the controllers 206A, 206B, the conductive members 390, and the wires 330.

During assembly of the intraluminal device 102, the conductors 332 and the conductive members 390 can be mechanically and electrically coupled separately from other components of the intraluminal device 102. Similarly, the conductive members 390 and the guide member 350 can be mechanically and electrically coupled separately from other components of the intraluminal device 102. For example, the conductors 332 and the conductive members 390, and/or the conductive members 390 and the guide member 350, can be manually coupled, such as a by a human operator, or coupled in an automated manner by a machine configured for such assembly processes. Coupling the conductors 332 and the conductive members 390, and/or the conductive members 390 and the guide member 350, apart from the imaging assembly 110 can increase efficiency of the manufacturing process compared to extant methods of individually aligning and welding/soldering individual conductors 332 and the conductive pads 322.

The guide member 350 can be an annularly shaped component that is positioned around the proximal flange 234 of the support member 230. The guide member 350 can be positioned proximally of the stand 244 and distally of the proximal end of the flange 234. In some instances, the guide member 350 can be press fit, interference fit, and/or otherwise engagingly fit around the flange 234. In some embodiments, adhesive(s) can be used to use to affix the guide member 350 to the flange 234.

The conductor interface 320 is wrapped in a semi-circular/semi-cylindrical manner around the guide member 350. In that regard, FIG. 5 illustrates the conductor interface 320 in a flat configuration, prior to being wrapped around the guide member 350. FIG. 6 illustrates the conductor interface 320 after it is wrapped around the guide member 350. When the conductor interface 320 is wrapped around the guide member 350, conductive pads 322 of the conductor interface 320 directly or indirectly contact and establish electrical communication with the conductive members 390. Adhesive(s) can be used to mechanically couple the conductor interface 320 in the semi-circular configuration around the guide member 350.

The conductor interface 320 extends proximally from the proximal portion 222 of the flex circuit 214. The features of the conductor interface 320 can be similar to those of the conductor interface 220 (FIG. 2). The conductor interface 320 includes the conductive pads 322. The conductive pads 322 are directly or indirectly in electrical communication with one or more components of the imaging assembly 110 such as the controllers 206A, 206B and/or the transducers 212 (FIG. 2). For example, the conductive traces 216 can facilitate communication between conductive pads 322, the controllers 206A, 206B, and/or the transducers 212. In some embodiments, the conductive pads 322 and the conductive traces 216 are similarly sized and shaped and/or formed of similar conductive materials.

In some embodiments, the conductive traces 216 extend within the flex circuit 214, and the conductive pads 322 are disposed on a top and/or a bottom surface of the flex circuit 214. In FIG. 6, for example, the conductive pads extend within the flex circuit 214 and are disposed on a bottom surface of the flex circuit 214 that is adjacent to guide member 350 and the conductive members 390. In some embodiments, the conductive pads 322 are sized and shaped to facilitate coupling to the conductors 332 of the wires 330 via the conductive members 390. For example, the conductive pads 322 can be relatively wider than the conductive traces 216. Accordingly, the conductive pads 322 can more easily make contact with the conductive members 390 to establish electrical communication between the cable 112, the controllers 206A, 206B, and/or the transducers 212. In some embodiments, the conductive members 390 can be soldered and/or welded to conductive pads 322. In some embodiments, adhesive(s) are used to affix the conductive members 390 and the conductive pads 322. The adhesive(s) may be electrically conductive to facilitate electrical communication between the conductive members 390 and the conductive pads 322.

In some embodiments, the guide member 350 and the conductive elements facilitate electrical communication between the cable 112 and the imaging assembly 110 without any welded/soldered components. For example, the conductive pads 322, the conductors 332, the guide member 350, the conductive members 390 can be mechanically coupled via adhesive(s) and/or any suitable engaging fit. The adhesive(s), the conductive pads 322, the conductors 332, and the guide member 350 can be formed of and/or include conductive materials to allow electrical communication without soldering/welding. Accordingly, the manufacturing components and process described herein can advantageously avoid a step that is currently performed to electrically and mechanically couple the wires 330 and the imaging assembly 110. In some embodiments, some components may be soldered/welded, which still achieves better manufacturing efficiency relative to some extant processes.

A proximal outer member 340 is illustrated in FIG. 5. The outer member 340 may be a flexible elongate member similar to the outer member 254 (FIG. 4). The outer member 340 may be positioned around the conductor interface 320 and the guide member 350 in some embodiments. The wires 330 of the cable 112 may extend along the length of the intraluminal device 102 within the outer member 340. In some instances, the wires 330 extend along the length of the intraluminal device 102 between the outer member 340 and an inner member similar to the inner member 256 (FIG. 4).

FIG. 8 is a diagrammatic perspective view of the guide member 350. The guide member 350 can be made of a non-conductive material such as a plastic or polymer. For example, guide member 350 can be made of polyimide, polyester, polyethylene napthalate, polyetherimide, poly ether ketone (PEEK) and/or other suitable materials. In the illustrated embodiment, the guide member 350 is cylindrically and annularly shaped. A proximal surface 351 of the guide member 350 is oriented in a proximal direction, and a distal surface 353 is oriented in a distal direction (FIGS. 7 and 8). The guide member 350 includes an inner surface 354 and an outer surface 356. While the inner surface 354 and the outer surface 356 are shown as circularly/cylindrically shaped, it is understood that the inner surface 354 and the outer surface 356 can have any suitable shape, including a polygon, an ellipse, etc. The inner diameter 364 associated with the inner surface 354 can be any suitable value suitable for mating (e.g., via press-fit, gluing, or other suitable technique). The inner surface 354 defines a lumen 355. As shown in FIGS. 5, 6, and 7, inner diameter 364 may be sized and shaped such that the proximal flange 234 extends through the lumen 355 such that the guide member 350 is positioned around the proximal flange 234. In some embodiments, the inner surface 354 may directly contact an outer surface of the proximal flange 234. In some embodiments, adhesive can be disposed between the inner surface 354 and the outer surface of the proximal flange 234.

The outer diameter 366 associated with the outer surface 356 can be any suitable value. In some embodiments, the dimensions of the guide member 350, such as the outer diameter 366, may be selected based on one or more dimensions of components of the intraluminal device 102 that mate, adjoin, contact, and/or otherwise couple with the imaging assembly 110. For example, the dimensions of the guide member 350 can be selected on the dimensions of the flex circuit 214, the support member 230, the inner member 256, the outer member 254, the distal member 252, and/or other components. In some embodiments, the outer diameter 366 can be selected based on the size of the intraluminal device 102, which can be between approximately 2 Fr and 12 Fr, for example. The outer diameter 366 may be selected such that the outer diameter of the assembled proximal portion of the imaging assembly 110 is less than or equal to the outer diameter of more distal portions of the outer diameter of the imaging assembly 110. That is, the guide member 350 and/or the conductive members 390 allow the imaging assembly 110 to achieve a relatively small diameter, facilitating efficient traversal through tortuous vessels. In some embodiments, the outer surface 356 may directly contact a bottom surface of the conductor interface 320. In some embodiments, adhesive can be disposed between the outer surface 356 and the bottom surface of the conductor interface 320.

A plurality of recesses 352 are formed in the guide member 350. The recesses 352 include an inner surface 358. The number of recesses 352 can be selected based on the number of wires 330 in the electrical cable 112. In the illustrated embodiment, the guide member 350 includes seven recesses 352. It is understood that in other embodiments, the guide member 350 may have any suitable number of wires 330, including two, three, four, five, six, seven, eight, or more. The recesses 352 can be have any suitable shape. As shown in FIG. 8, for example, the recesses 352 can be U-shaped. In other embodiments, the recesses 352 can be V-shaped, circular, square, rectangular, polygonal, ellipsoidal, etc.

The recesses 352 can be positioned around a circumference of the guide member 350. The recesses 352 can be spaced apart by any suitable distance. For example, the wires 330 and the corresponding recesses 352 can be aligned on the power and/or wiring requirements of the intraluminal device 102. In some embodiments, the electrical cable 112 can include between two and ten wires, for example. In some embodiments, the recesses 352 are distributed around the complete circumference of the guide member 350. In some embodiments, the recesses 352 are positioned in one or more areas of the guide member 350. For example, the recesses 352 can be positioned in an upper or lower half of the guide member 350, a quadrant, and/or any other suitable subdivision of the guide member 350.

The recesses 352 extend a depth 362 from the outer surface 356 radially and inwardly towards the inner surface 354. The depth 362 can be any suitable value. The recesses 352 have a width 369 in a direction perpendicular to longitudinal axis LA. The width 369 can be any suitable value. The guide member 350 and the recesses 352 have a length 368 in a direction parallel to the longitudinal axis LA. The length 368 can be any suitable value.

FIG. 9 is a diagrammatic perspective view of the conductive member 390. The imaging assembly can include any suitable number of conductive members 390, including two, three, four, five, six, seven, eight, or more. The conductive members 390 can be formed of a conductive material, such as a metal or metal alloy, including copper, gold, aluminum, silver, tantalum, nickel, tin, combinations thereof, and/or other suitable materials. In some embodiments, the conductive members 390 are formed of a single material. In some embodiments, the conductive members 390 can be plated. For example, the conductive members 390 can be formed of a first material that is coated with a second material with better conductive properties than the first material. For example, the conductive members 390 can be formed of nickel or copper, with gold plating.

The conductive members 390 can be have any suitable shape. As shown in FIG. 9, for example, the conductive members 390 can be U-shaped. In other embodiments, the conductive members 390 can be V-shaped, circular, square, rectangular, polygonal, ellipsoidal, etc. In some embodiments, the conductive members 390 can be a spring that is configured to compress and extend. The wires 330 can be received within the spring to contact and establish electrical communication. Generally, the recesses 352 and the conductive members 390 are correspondingly sized and shaped such that the conductive members 390 are received within the recesses 352 when the imaging device 102 is assembled. The conductive members 390 can be characterized as plugs in some instances in that they are received within the recesses 352. A side surface 393 and a rounded end 389 of the conductive member 390 contact the inner surface 358 of the recesses 352 of the guide member 350. A top surface 398 of the conductive member 390 contacts the conductive pad 322 (e.g., a bottom surface of the conductive pad 322) when the conductor interface 320 is wrapped around the guide member 350.

Bores 392 extends longitudinally through the conductive members 390 between a proximal surface 399a and a distal surface 399b (FIGS. 7 and 9). The proximal surface 399a of conductive member 390 is oriented in a proximal direction, and a distal surface 399b is oriented in a distal direction. The bores 392 can be defined by an inner surface 397 of the conductive members 390. The bores 392 are sized and shaped to receive the bare conductors 332 and/or the insulating layer 334 of the wires 330. A diameter 391 of the bores 392 can be any suitable value. For example, a diameter of the bores 392 can be greater than or equal to the diameter of the conductors 332 in some embodiments. The conductors 332 can directly or indirectly contact the inner surface 397 to establish electrical communication between the wires 330 and the conductive members 390.

A height 394 of the conductive member 390 can be any suitable value. The height 394 of the conductive members 390 and the depth 362 of the recesses 352 (FIG. 8) can be related. For example, the depth 362 can be less than or equal to the height 394 in some embodiments. In some embodiments, the height 394 of the conductive members 390 extends beyond the outer surface 356 of the guide member 350 when the conductive members 390 are positioned within the recesses 352. In such instances, the conductive members 390 form raised portions that contact the conductive pads 322. The raised portions may allow for easier alignment of the conductive pads 322 and the conductive members 390. In some embodiments, the height 394 of the conductive members 390 is selected such that the top surfaces 398 and the outer surface 356 of the guide member 350 form a substantially continuous surface. In such embodiments, the conductor interface 320 can be smoothly wrapped around the guide member 350.

The conductive members 390 have a length 395 in a direction parallel to the longitudinal axis LA. The length 395 can be any suitable value. The length 395 of the conductive members 390 and the length 368 of the guide member 350 and the recesses 352 (FIG. 8) can be related. For example, the length 368 can be equal to the length 395 in some embodiments. A length of the conductive pads 322 can also be related to the length 395 of the conductive member 390. For example, the length of the conductive pads 322 can be greater than or equal to the length 395 such that the entire length 395 of the conductive members 390 contacts the length of the conductive pads 322.

The conductive members 390 have a width 396 in a direction perpendicular to the longitudinal axis LA. The width 396 can be any suitable value. The width 396 of the conductive members 390 and the width 369 of the recesses 352 can be related (FIG. 8). For example, the width 369 can be equal to the width 396 in some embodiments. The width of the conductive pads 322 and the width 396 of the conductive members 390 can be related. For example, the width of the conductive pads 322 can be greater than or equal to the width of the conductive members 390 such that the entire width of the conductive members 390 contacts the width of the conductive pads 322.

Generally, one or more dimensions the dimensions of guide member 350 and/or the conductive members 390 can be based on the size, such as a diameter, of the wires 330 and/or the conductors 332. Accordingly, the dimensions of the guide member 350 and/or the conductive members 390 can be adjusted and/or selected to correspond to the size of the wires 330 and/or the conductors 332. The dimensions of the guide member 350 and/or the conductive members 390 can be selected based on how the wires 330 and/or the conductors 332 are aligned and/or the connection of the wires/conductors to the conductive members 390/guide member 350.

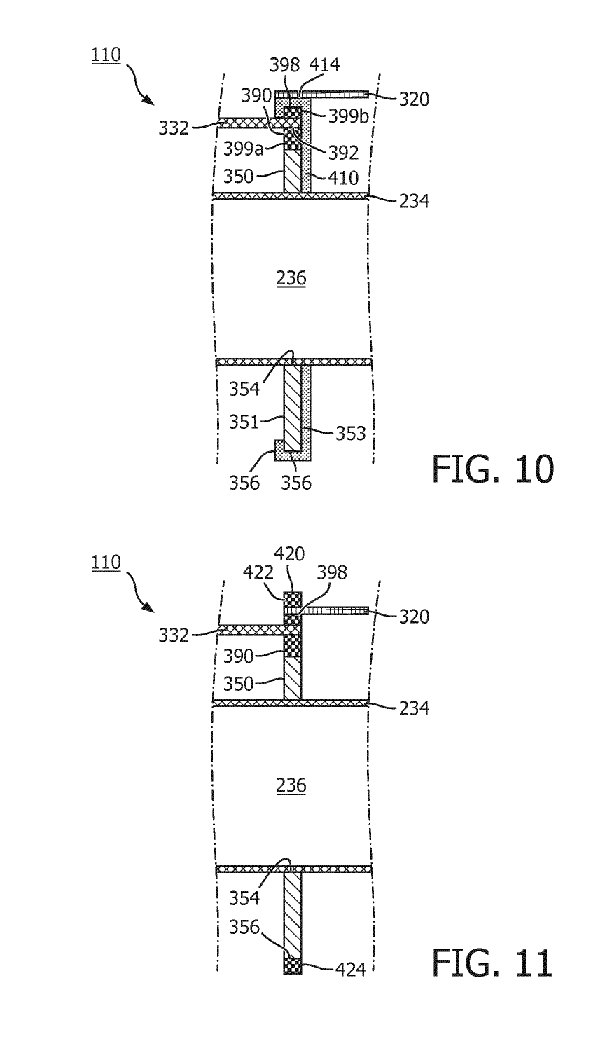

FIG. 10 is a diagrammatic cross-sectional side view of the imaging assembly 110. The components illustrated in FIG. 10 are similar to those shown in FIG. 7. FIG. 10 additionally includes an engagement mechanism or member 410 configured to maintain the conductive members 390 within the recesses 352 of the guide member 350. The engagement or attachment mechanism 410 can be a generally annularly shaped component that is positioned adjacent to and/or in contact with the guide member 350, the conductive members 390, and/or the conductor interface 320. In the illustrated embodiment, the engagement mechanism 410 can be disposed adjacent to and/or in contact with the distal surface 353 of the guide member 350 and the distal surface 399b of the conductive members 390. The engagement mechanism 410 is positioned around the guide member 350 and the conductive members 390. A portion 414 of the engagement mechanism 410 extends around the outer surface 356 of the guide member 350 and the conductive members 390. A locking feature 412 of the engagement mechanism 410 maintains the engagement mechanism 410 in contact with guide member 350 and the conductive members 390. The portion 414 mechanically urges the conductive members radially into the recesses 352 of the guide member 350. The portion 414 may include a via to facilitate electrical communication between the conductive members 390 and the conductor interface 320.

FIG. 11 is a diagrammatic cross-sectional side view of the imaging assembly 110. The components illustrated in FIG. 11 are similar to those shown in FIGS. 7 and 10. FIG. 11 includes an engagement or attachment mechanism 420 configured to maintain the conductive members 390 within the recesses 352 of the guide member 350. The engagement mechanism or member 420 also maintains contact between the conductive pads 322 of the conductor interface and the conductive members 390. The engagement mechanism 420 can be a generally annularly shaped component. A portion 422 of the engagement mechanism 420 is positioned adjacent to and/or in contact with the conductor interface 320. A portion 424 of the engagement mechanism 420 is positioned adjacent to and/or in contact with the outer surface 356 of the guide member 350. The engagement mechanism 420 is positioned around the guide member 350, the conductive members 390, and the conductor interface 320. The engagement mechanism 420 mechanically urges the conductive members radially into the recesses 352 of the guide member 350 via contact with the conductor interface 320 and the outer surface 356.

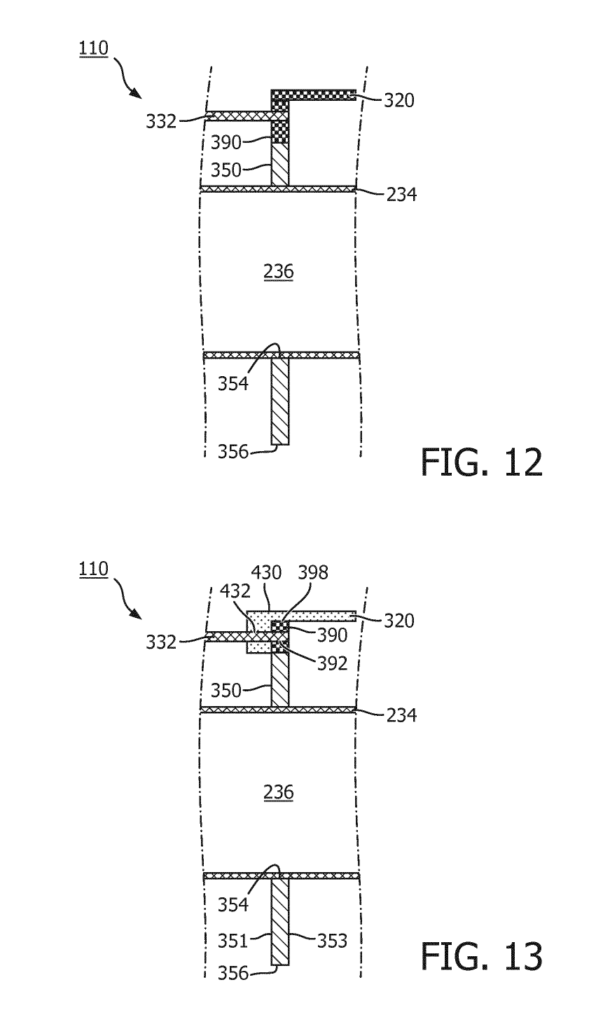

FIG. 12 is a diagrammatic cross-sectional side view of the imaging assembly 110. The components illustrated in FIG. 12 are similar to those shown in FIG. 7. In the embodiment of FIG. 11, the conductive members 390 are attached to the conductor interface 320. For example, the conductive members 390 may be integrally formed with the conductor interface 320. In other instances, the conductive members 390 and the conductor interface 320 may be distinct components that are selectively attached to the conductor interface 320, such as by adhesive(s). The conductive members 390 and the conductor interface 320 are in electrical communication. During assembly, the conductors 332 can be electrically and mechanically coupled to the conductive members 390, which are then coupled to the guide member 350 along with the conductor interface 320. By having the conductive members 390 and the conductor interface 320 attached, manufacturing efficiency may be increased by reducing the total number of distinct components that are coupled.

FIG. 13 is a diagrammatic cross-sectional side view of the imaging assembly 110. The components illustrated in FIG. 13 are similar to those shown in FIGS. 7 and 12. In the embodiment of FIG. 13, the imaging assembly 110 includes an attachment member 430 that is integrally formed with the conductor interface 320. The attachment member 430 can be at least partially positioned adjacent to and/or in contact with the conductive members 390, such as the top surface 398. The attachment member 430 may be unitary component that extends in a semicircular manner along a circumference of the guide member 350. In some embodiments, the conductor interface is coupled to a plurality of attachment members 430. The number of attachment members 430 can correspond to the number of conductors 332. The attachment member(s) 430 includes bores 432 sized and shaped to receive the conductor 332. The bores 432 can be aligned with respective bores 392 of the conductive members 390 such that the conductor 332 extends through the bores 392 and 432. The conductors 332 may be in electrical communication with the conductor interface 320 via the attachment members 430 and/or the conductive members 390. The conductive members 390 and the conductor interface 320 are in electrical communication. During assembly, the conductors 332 can be electrically and mechanically coupled to the conductive members 390 and the attachment members 430 in a single step. Manufacturing efficiency may be increased by reducing the total number of distinct components that are coupled in order to establish electrical communication between the conductors 332 and the conductor interface 320.

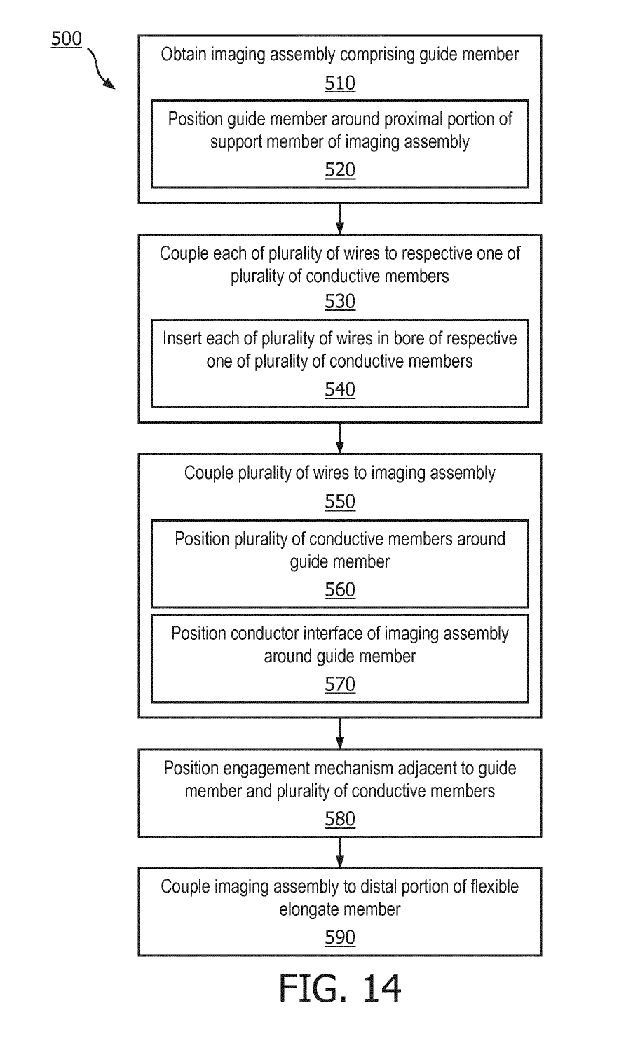

FIG. 14 is a flow diagram of a method 500 of assembling an intraluminal imaging device, as described herein. It is understood that the steps of method 500 may be performed in a different order than shown in FIG. 14, additional steps can be provided before, during, and after the steps, and/or some of the steps described can be replaced or eliminated in other embodiments. The steps of the method 500 can be carried out by a manufacturer of the intraluminal imaging device.

At step 510, the method 500 includes obtaining an imaging assembly including a guide member. The imaging assembly can include a support member and a flex circuit positioned around the support member. The guide member can be annularly-shaped. In some embodiments, step 510 includes positioning the guide member around a proximal portion of the support member (step 520).

At step 530, the method 500 includes coupling each of a plurality of wires to a respective one of a plurality of conductive members. Each of the plurality of conductive members can include a bore. In some embodiments, step 530 can include inserting each of the plurality of wires in the bore of the respective one of the plurality of conductive members (step 540).

At step 550, the method 500 includes coupling the plurality of wires to the imaging assembly. Step 550 can include positioning the plurality of conductive members around the guide member (step 560). The guide member can include a plurality of recesses. Step 560 can include positioning each of the plurality of conductive members within a respective one of the plurality of recesses.

The imaging assembly can include a conductor interface. In some embodiments, at step 570, the method 500 can include positioning the conductor interface around the guide member. The conductor interface can include a plurality of conductive pads. Step 570 can include contacting each of the plurality of conductive pads of the conductor interface with a respective one of the plurality of conductive members of the guide member.

At step 580, the method 500 includes positioning an engagement mechanism adjacent to the guide member and the plurality of conductive members to maintain to plurality of conductive members within the plurality of recesses of the guide member.

At step 590, the method 500 includes coupling the imaging assembly to a distal portion of a flexible elongate member. For example, step 590 can include positioning the flex circuit around a support member to form an imaging assembly of the intraluminal device. The flex circuit may initially be in a flat configuration. Step 590 can include transitioning at least a portion of the flex circuit into a rolled configuration around the support member. The flex circuit can be positioned around the support member such that the inner diameter of the flex circuit contacts a backing material disposed between the support member and the flex circuit. The method 500 may include securing the flex circuit to the support member using one or more adhesives. The method 500 may also include curing the backing material, such as by using heat or light. The method 500 includes coupling the imaging assembly to one or more distal members and one or more proximal members to form the intraluminal device. In that regard, the distal member(s) and/or proximal member(s) can be coupled to the support member and/or the flex circuit. The one or more proximal members may be flexible elongate members (e.g., an inner member and/or an outer member) forming a length of the intraluminal device. The imaging assembly may be positioned at a distal portion of the intraluminal device. The distal member defines a distal-most end of the intraluminal imaging device. The method 500 can include introducing adhesive to affix the flex circuit and the support member and/or other components of the intraluminal imaging device.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. An intraluminal imaging device, comprising:
a flexible elongate member configured for insertion into a vessel of a patient, the flexible elongate member including a proximal portion and a distal portion;
an imaging assembly disposed at the distal portion of the flexible elongate member;
a plurality of wires extending along a length of the flexible elongate member and in communication with the imaging assembly; and
a guide member being at a proximal portion of the imaging assembly and comprising a plurality of conductive members, wherein the plurality of wires are in communication with the imaging assembly via the plurality of conductive members.

2. The device of claim 1, wherein
the imaging assembly comprises a conductor interface; and
the conductor interface is positioned around at least a portion of the guide member.

3. (canceled)

4. The device of claim 2, wherein:
the imaging assembly comprises a support member and a flex circuit positioned at least partially around the support member, and
the conductor interface extends from a proximal portion of the flex circuit.

5. The device of claim 4, wherein the guide member is annular.

6. The device of claim 5, wherein the guide member is positioned around a proximal portion of the support member.

7. The device of claim 1, wherein each of the plurality of conductive members comprises a bore configured to receive at least one of the plurality of wires.

8. The device of claim 7, wherein each of the plurality of wires extends through the bore of a respective one of the plurality of conductive members.

9. The device of claim 1, wherein the guide member comprises a plurality of recesses, each of the plurality of recesses configured to receive a respective one of the plurality of conductive members.

10. The device of claim 9, further comprising an engagement mechanism positioned adjacent to the guide member and the plurality of conductive members, and arranged to maintain to plurality of conductive members within the plurality of recesses of the guide member.

11. The device of claim 1, wherein the plurality of conductive members are positioned around a circumference of the guide member.

12. The device of claim 1, wherein the guide member comprises non-conductive material.

13. A method of assembling an intraluminal imaging device, the method comprising:
obtaining an imaging assembly and a guide member, the imaging assembly comprising a conductor interface including a plurality of conductive pads;
coupling each of a plurality of wires to a respective one of a plurality of conductive members; and
coupling each of the plurality of wires to a respective one of the plurality of conductive pads of the imaging assembly by positioning the plurality of conductive members around the guide member.

14. The method of claim 13, further comprising
positioning the conductive pads of conductor interface around the guide member.

15. The method of claim 14, wherein the positioning the conductor interface around the guide member comprises contacting each of the plurality of conductive pads of the conductor interface with a respective one of the plurality of conductive members.

16. The method of claim 13, wherein the imaging assembly comprises a support member and a flex circuit positioned around the support member, wherein the guide member is annular, and wherein the method further comprises
positioning the guide member around a proximal portion of the support member.

17. The method of claim 13, wherein the guide member comprises a plurality of recesses, wherein the positioning the plurality of conductive members around the guide member comprises positioning each of the plurality of conductive members within a respective one of the plurality of recesses.

18. The method of claim 13, furthering comprising:
positioning an engagement mechanism adjacent to the guide member and the plurality of conductive members to maintain to plurality of conductive members within the plurality of recesses of the guide member.

19. The method of claim 13, wherein each of the plurality of conductive members comprises a bore, and wherein the coupling each of a plurality of wires to a respective one of a plurality of conductive members comprises inserting each of the plurality of wires into the bore of the respective one of the plurality of conductive members.
